(19) United States
(12) Patent Application Publication
(10) Pub. No.: US 2014/0236136 A1
(43) Pub. Date: Aug. 21, 2014

(54) HAIR REMOVAL BASED ON IPL

(71) Applicant: EPILADY 2000, LLC., Hatzor Haglilit (IL)

(72) Inventor: Oren AHARON, Haifa (IL)

(73) Assignee: EPILADY 2000, LLC., Hatzor Haglilit (IL)

(21) Appl. No.: 14/185,868

(22) Filed: Feb. 20, 2014

(30) Foreign Application Priority Data
Feb. 20, 2013 (IL) 224821

(51) Int. Cl. A61B 18/18

(52) U.S. Cl. CPC A61B 18/18
USPC 606/9

(57) ABSTRACT

A device and method of hair removal treatment is disclosed, The device includes a combination of two light sources one an IPL and second an incandescent lamp type, halogen or others. The two light sources are applied simultaneously or sequentially to the treated area. The present invention relates to a method for hair removal treatment where multiple light sources are combined together and include at least one pulsed arc lamp, and are operated simultaneously or sequentially to treat a selected skin area. The incandescent light energy will raise the overall skin temperature while the arc lamp will selectively ablate the hair follicle with minimum collateral damage.

### CROSS-REFERENCE TO RELATED APPLICATIONS

This application claims the benefit of Israeli Application No. 224821, filed Feb. 20, 2013, in the Israeli Patent Office. All disclosures of the document(s) named above are incorporated herein by reference.

### BACKGROUND OF THE INVENTION

1. Field of the Invention

The invention relates to a device for performing aesthetic treatments such as acne treatment, wrinkle removal, hair removal and other application based on light treatment. The system may comprise of a dual source light system one incandescent and second IPL.

2. Description of the Related Art

Known state of art existing methods are characterized by usage of a relatively large light source with a specific wavelength range, which floods a large area, said light source is capable of selective treatment by photothermolysis. For example hair removal is based on the principle of selectively heating and destroying the hair follicle while avoiding significant damage to surrounding skin or tissue. Hair follicles are selected by photothermolysis an effect based on the fact that hair absorbs great amounts of infrared light, due to its darker color when compared with surrounding skin tissue similar mechanism will work for other treatment such as stains and veins. Hair is thus automatically selected by the light since it has a darker color and thus higher absorption coefficient. Current technology is based on IPL lamp which emits high energy light pulses to perform the thermolysis treatment. The main drawback of this technology is that high energy light pulses are characterized by having most of light energy in the visible range which is useless for hair removal purposes. Hair removal is performed by the IR range of light spectrum. On the other hand other light sources such as an incandescent lamp, due to their lower black body temperature, emit most of their energy in the IR region of optical spectrum.

In known hair removal systems treatment is performed by flooding with light a relatively large area with a high intensity pulsed light. A typical system of a device for dermatological treatment is intended to perform dermatological treatment by intense pulses of light radiated over large skin areas. The treatment selection is performed by chromatic characteristics the skin or hair follicles and selection between treated and not to be treated areas is performed by the light source wavelength selection in a process called photo thermolysis or wavelength depended light absorption.

Light is absorbed by dark objects, so light energy primarily in the IR spectral region can be absorbed by dark material in the skin, but with higher speed and intensity. This dark target matter, or chromophore, can be naturally-occurring or artificially introduced. The main drawback of this procedure is that large area of skin are unnecessarily exposed to high levels of intense pulsed light with potential adverse results.

For example conventional laser hair removal systems rely on flooding large areas with high intensity light, and removal is performed at a wavelength that will not damage the human tissue, where the light intensity will be transmitted by the skin to the follicle depth and destroy the follicle by chromo-thermolysis.

Since an incandescent lamp emits most of its energy in the IR region, it is potentially a perfect candidate for hair removal, but unfortunately, due to its incandescent filament, its rise and fall time when activated is very slow. On the other hand, an arc lamp has fast rise and fall time, thereby permitting very fast pulsing with low percentage of IR emitting. A combination between the two sources will create a better solution for hair removal purposes.

### SUMMARY OF THE INVENTION

Advantages over the Existing Systems

The apparatus uniqueness is in the fact that it achieves and sometimes overcomes the performance of the systems existing in the market for the above procedures, while using less pulsed light energy for hair removal.

The treatment apparatus is based on a multiple light sources of incandescent and arc lamps working together for hair removal purposes. The internal light source structure enables the same to operate with high peak intensity for the effective treatment, while the emission spectrum remains mostly in the near infra-red region.

It is another aspect of this invention to offer a different method based on combination of two light sources one an incandescent lamp with long light pulses and second being an intense pulse light (IPL) with short pulses. This combination will preheat the treated area with incandescent lamp and by irradiating using IPL the hair follicle ablation will occur.

Many disadvantages of prior art aesthetic treatment devices are advantageously solved in present invention. A partial list will follow:

In prior art systems a high power light pulse is applied while most of its energy is filtered out to allow only IR light to penetrate and reach the skin level.

In prior art treatment is performed only by intense pulsed light with very high peak levels. It is further the object of this invention to lower the light peak level while increasing the total energy irradiation on treated areas.

It is an aspect of the present invention to provide a treatment device capable of hair removal treatment by a combination of an incandescent lamp providing long pulses and low peak power and an arc lamp providing IPL pulses with high momentary power.

There is provided in accordance with preferred embodiment an aesthetic treatment device the device comprising of:

Multiple treating light sources with at least one incandescent and another arc lamp.

Optical reflector for directing light output to treated area.

A pulse generator to control the treating light sources power output.

Mechanical enclosure for optical device.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic presentation of proposed aesthetic treatment device according to an embodiment of the present invention; and

FIG. 2 is a schematic representation of a hand piece encapsulating light sources and controlling electronics of the aesthetic treatment device shown in FIG. 1.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

The present invention discloses an aesthetic treatment device enabling application of multiple light sources with at least one incandescent and one arc lamp.

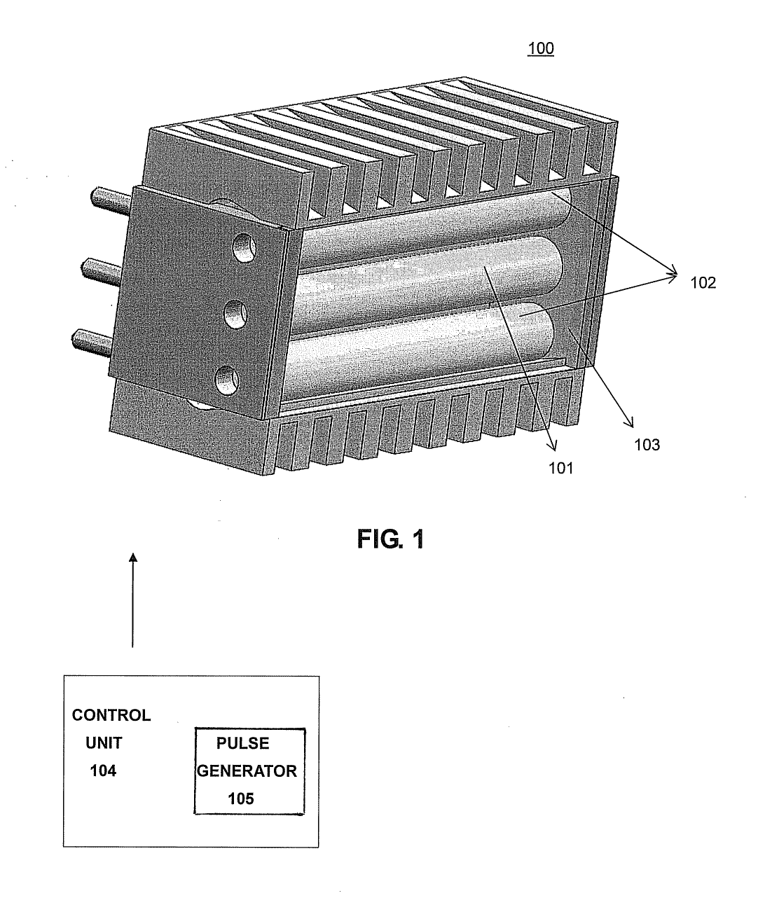

FIG. 1 is a schematic presentation of an aesthetic treatment device 100 enabling treatment and hair removal by exposing a treated area to a combination of light sources, and in this embodiment, one being at least one type of light source, for example, an incandescent lamp 102 and the second being a different type of light source, such as an intense pulse light (IPL) lamp 101, for example, an arc lamp. In this embodiment, there are two tubular incandescent lamps 102, one immediately above the arc lamp 101 and one immediately below the arc lamp 101, although there may be only one incandescent lamp 102 and there may be more than 2 such lamps. Here, the incandescent lamps 102 and the arc lamp 101 are arranged parallel to each other and in a vertical plane one above the other. Exposure time is different for the two sources, where the incandescent lamps 102 expose the treated area to a relative long pulse in the second range, while the arc lamp 101 flashes in the milliseconds range. The long pulse is intended to elevate the temperature of both skin and hair while the flash will ablate the hair follicle by thermolisys. An optical reflector directs the emitted light to a treatment area of the skin of a patient.

Here, also by way of example, the arc lamp 101 is also tubular and is singular in number, although variations in both the shape and number of arc lamps are possible. The radiating area of the skin is protected by a window 103.

A control unit 104 controls light parameters of the arc lamp 101 and the incandescent lamps 102 to be applied to skin. Control is performed through a pulse generator 105 of the control unit 104 and the intensity duration is adjusted as required for a specific aesthetic treatment.

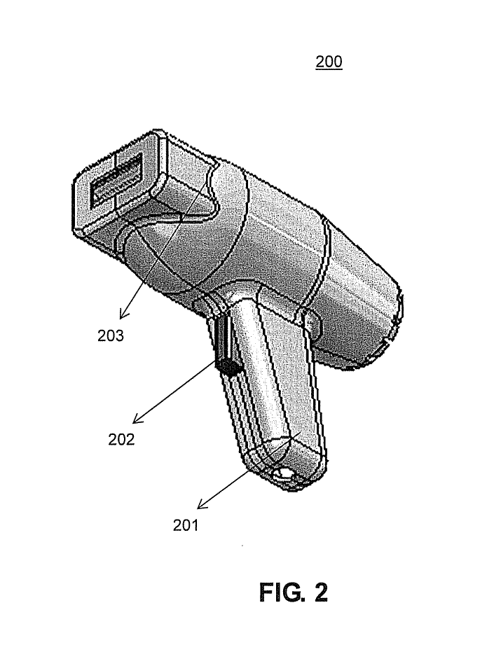

FIG. 2 is a schematic representation of a hand piece 200, with a hand piece shell 201 encapsulating the light sources 101 and 102, the window 103, the control unit 104 and the pulse generator 105. An activating button 202 determines when the specific aesthetic treatment is to be performed, and the light from the arc lamp 101 and the incandescent lamps 102 are output through an aperture 203.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. An aesthetic treatment device, comprising:
multiple treating light sources with at least one incandescent lamp and an intense pulse light (IPL) lamp to output power;
an optical reflector for directing light output to a treatment area;
a pulse generator to control the treating light sources power output through pulse generation adjustment; and
a mechanical enclosure for containing the multiple treating light sources, the optical reflector and the pulse generator.
